(19) United States
(12) Patent Application Publication
(10) Pub. No.: US 2025/0177186 A1
(43) Pub. Date: Jun. 5, 2025

(54) CANNISTER-FREE, VACUUM-ASSISTED EVACUATION OF EXTERNAL CATHETERS

(71) Applicant: Stephen L. Rawlins, Lehi, UT (US)

(72) Inventor: Stephen L. Rawlins, Lehi, UT (US)

(21) Appl. No.: 18/530,048

(22) Filed: Dec. 5, 2023

(51) Int. Cl. A61F 5/453; A61F 5/455; A61M 1/00

(52) U.S. Cl. CPC A61F 5/453; A61F 5/455; A61M 1/80

(57) ABSTRACT

An apparatus for collecting urine from a user includes a urinary catheter comprising an outlet to enable liquid to exit therefrom. A first end of a transport channel is operable coupled to the outlet. A pump module, which may be cannister free, is coupled to a second end of the transport channel and is configured to generate suction in the transport channel to draw the liquid into the pump module. The pump module expels the liquid into an output channel. The pump module is configured to draw air as well as the liquid through the transport channel. In certain embodiments, an air entry module is placed at or near the outlet to enable air to enter the transport channel.

### BACKGROUND

### Field of the Invention

This invention relates to urinary catheters, and more particularly to external urinary catheters that can be evacuated with the assistance of a vacuum device.

### Background of the Invention

Urinary catheters are used extensively in the medical field to empty a patient's bladder into a bag or other container. Such catheters may be used for health issues such as urinary incontinence, urinary retention, surgery, immobility, or the like. Urinary catheters come in various different types including indwelling catheters, external catheters, intermittent catheters, and the like. They may also be specifically designed for male or female use. However, whatever type a catheter may be, they are almost always designed for use in professional or commercial settings. They are also typically designed to be disposable and thus are intended to be replaced on a regular basis. What is missing in the marketplace is a urinary catheter that is geared toward a more casual user in a non-medical setting.

### SUMMARY

The invention has been developed in response to the present state of the art and, in particular, in response to the problems and needs in the art that have not yet been fully solved by currently available apparatus and methods. Accordingly, apparatus and methods in accordance with the invention have been developed to collect urine from a user. The features and advantages of the invention will become more fully apparent from the following description and appended claims, or may be learned by practice of the invention as set forth hereinafter.

Consistent with the foregoing, an apparatus for collecting urine from a user includes a urinary catheter comprising an outlet to enable liquid to exit therefrom. A first end of a transport channel is operable coupled to the outlet. A pump module, which may be cannister free, is coupled to a second end of the transport channel and is configured to generate suction in the transport channel to draw the liquid into the pump module. The pump module expels the liquid into an output channel. The pump module is configured to draw air as well as the liquid through the transport channel. In certain embodiments, an air entry module is placed at or near the outlet to enable air to enter the transport channel in a regulated manner.

### BRIEF DESCRIPTION OF THE DRAWINGS

In order that the advantages of the invention will be readily understood, a more particular description of the invention briefly described above will be rendered by reference to specific embodiments illustrated in the appended drawings. Understanding that these drawings depict only typical embodiments of the invention and are not therefore to be considered limiting of its scope, the invention will be described and explained with additional specificity and detail through use of the accompanying drawings, in which:

FIG. 1 is a perspective view showing one embodiment of a urinary catheter in accordance with the invention, including an air entry module installed near an outlet thereof;

FIG. 2 is a perspective view showing the urinary catheter of FIG. 1, and more particularly showing air bubbles being pulled into the channel through the air entry module to assist with conveying urine through the channel;

FIG. 3 is perspective view of a cannister-free system including the urinary catheter and a pump module to evacuate urine from the urinary catheter;

FIG. 4 is a high-level block diagram showing various exemplary internal components of a pump module in accordance with the invention; and

FIG. 5 is perspective view of a cannister-free system including the urinary catheter and a pump module to evacuate urine from the urinary catheter into a receptacle.

### DETAILED DESCRIPTION

It will be readily understood that the components of the present invention, as generally described and illustrated in the Figures herein, could be arranged and designed in a wide variety of different configurations. Thus, the following more detailed description of the embodiments of the invention, as represented in the Figures, is not intended to limit the scope of the invention, as claimed, but is merely representative of certain examples of presently contemplated embodiments in accordance with the invention. The presently described embodiments will be best understood by reference to the drawings, wherein like parts are designated by like numerals throughout.

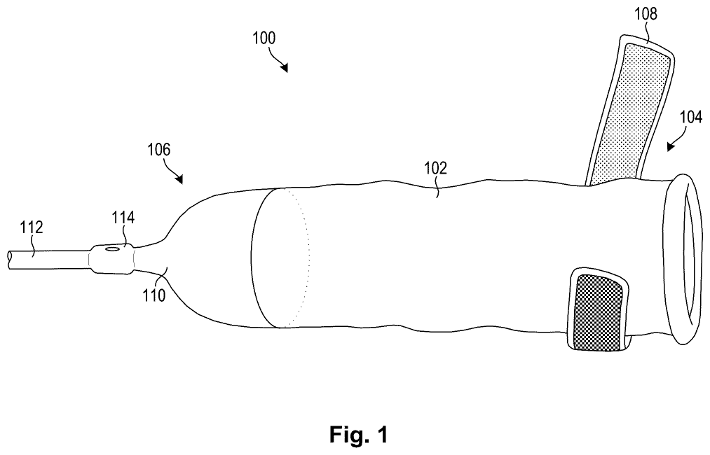

Referring to FIG. 1, as previously mentioned, urinary catheters are used extensively in the medical field to empty a patient's bladder into a bag or other container. Such catheters may be used for health issues such as urinary incontinence, urinary retention, surgery, immobility, or the like. Urinary catheters come in various different types, including indwelling catheters, external catheters, intermittent catheters, and the like. They may also be specifically designed for male or female use. However, whatever type a catheter may be, they are almost always designed for use in professional or commercial settings. They are also typically designed to be disposable and thus are intended to be replaced on a regular basis.

What is missing in the marketplace is a urinary catheter that is geared toward a more casual user for use in a non-medical setting. For example, a casual user could potentially use a urinary catheter in his or her home to urinate and thereby avoid having to get up in the middle of the night and interrupt the user's rest or sleep. This may be particularly useful with older users, users that have to urinate frequently due to a medical or other condition, or users that have impaired mobility. Other potential users may include campers, truckers, or workers that may not have convenient or frequent access to bathrooms. Ideally such a catheter would be inexpensive, easy to clean, and reusable to cut down on waste. Further needed are techniques to effectively evacuate the catheter of urine that go beyond simply relying on gravity.

FIG. 1 is a perspective view showing one embodiment of a urinary catheter 100 in accordance with the invention. The urinary catheter 100 shown in FIG. 1 may also be referred to as a “condom catheter 100”. As shown, the urinary catheter 100 includes a sheath portion 102 having a first end 104 and a second end 106. The first end 104 is configured to make an air and/or liquid tight seal with a penis of a user. The seal may be generated by a fit of the sheath portion 102 around the penis of a user and/or additional mechanisms, such as the illustrated strap 108 with hook and loop fasteners, that may be tightened around the penis of a user in order to enhance the seal of the sheath portion 102 around the penis.

The urinary catheter 100 may be referred to as a “condom catheter” since it may fit on the penis like a condom. The urinary catheter 100 may also be referred to as an external urinary catheter or a penile sheath catheter. The urinary catheter 100 may be made of any suitable material such as latex or silicon. It may also be provided in different sizes. Although the urinary catheter 100 is shown with a strap 108, the urinary catheter 100 may also be attached to a user with an adhesive, tape, or the like.

The second end 106 of the sheath portion 102 includes an outlet 110 that enables urine to exit the sheath portion 102. A urine transport channel 112 (e.g., a flexible tube, such as a flexible plastic, rubber, or silicone tube) may be operably coupled to this outlet 110 to enable the urine to be conveyed away from the urinary catheter 100. In some cases, the urine may flow away under the influence of gravity. This may be possible in applications where the user is standing and any collection area or chamber is below the level of the urinary catheter 100. Unfortunately, such gravity-based systems may, in certain cases, leave the penis bathed in urine. To prevent this from occurring, the urine may also be conveyed away from the urinary catheter 100 using suction, such as that generated by a pump or vacuum device as will be explained in more detail hereafter. This may be more useful in applications where the user is seated or laying down or the urine collection area or chamber is at or above the level of the urinary catheter 100. This may also more effectively evacuate urine from the urinary catheter 100 to leave a user more comfortable between urination events.

In order to facilitate conveyance of urine away from the urinary catheter 100, an air entry module 114 (e.g., a check valve, air throttle valve, etc.) may be coupled to the urine transport channel 112 at or near the outlet 110. This may enable air 115 to enter the urine transport channel 112 and urinary catheter 100 to replace urine that is drawn out of the urine transport channel 112 by the pump or vacuum device. The air entry module 114 may also in certain embodiments prevent air and/or liquids from exiting the urine transport channel 112 through the air entry module 114. Ideally, the air entry module 114 is placed as close to the outlet 110 as possible. For example, in certain embodiments, the air entry module 114 is placed within one inch of the outlet 110. In other embodiments, the air entry module 114 is placed within two inches of the outlet 110.

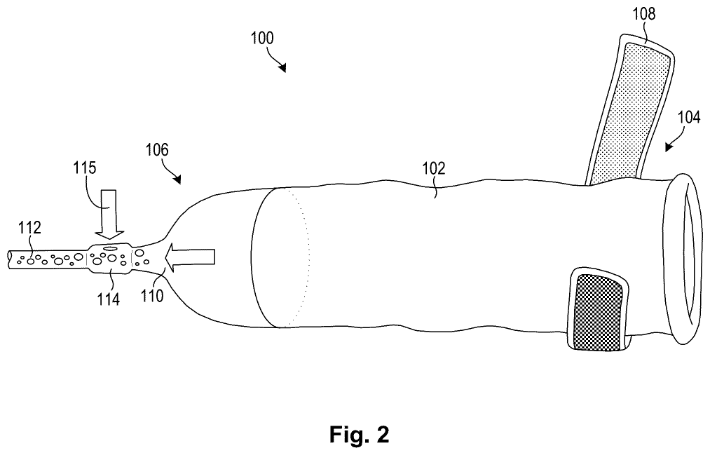

As shown in FIG. 2, as urine is drawn away from the urinary catheter 100 through the urine transport channel 112 using a pump or vacuum, air may enter the urine transport channel 112 through the air entry module 114. Because the sheath portion 102 essentially forms a sealed container with the penis, the air may replace the urine that is drawn out of the urinary catheter 100. In addition, air bubbles that enter through the air entry module 114 may travel up the urine transport channel 112 with the urine to assist in conveying the urine to a disposal area, such as to a toilet or other receptacle.

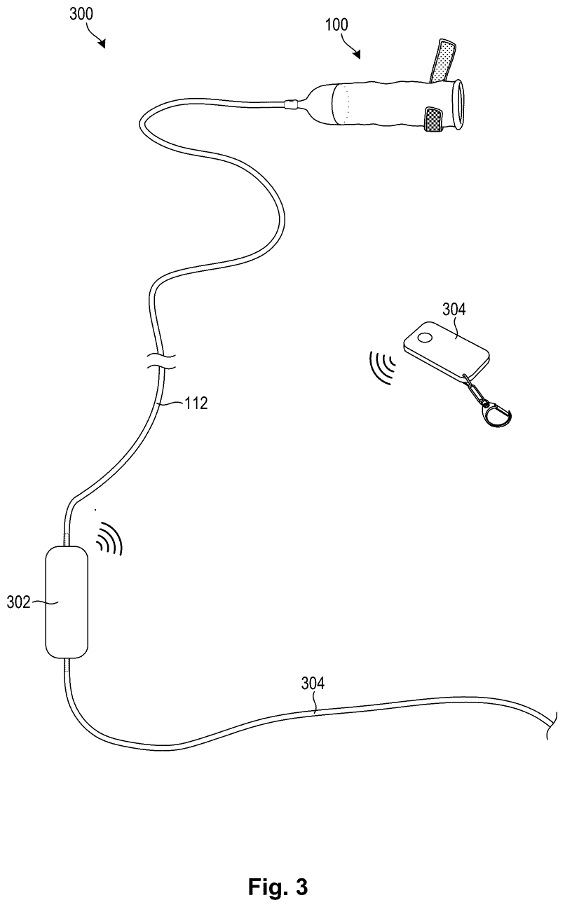

Referring to FIG. 3, one embodiment of a system 300 for collecting urine from a user is illustrated. As shown, the system 300 includes the urinary catheter 100 shown in FIGS. 1 and 2, and a pump module 302 configured to pull urine from the urinary catheter 100 and convey it to a disposal area. In the illustrated embodiment, when activated (i.e., turned on) the pump module 302 generates suction within the transport channel 112. This may pull urine and air bubbles into the transport channel 112 to evacuate the urinary catheter 100. In certain embodiments, the pump module 302 is configured to generate suction within the transport channel 112 as well as pump liquid from the transport channel 112 without utilizing a vacuum canister, as is typical with many vacuum devices. The liquid may be expelled into an outlet channel 304. The outlet channel 304 may be routed to a disposal area such as toilet or receptacle, as previously discussed.

Most liquid pumps are not designed to also function as vacuum devices. Rather, liquid pumps are primarily intended for the purpose of moving and pressurizing liquids, such as water or other fluids. They function by creating a flow of liquid from one location to another, often through the use of mechanical impellers or other mechanisms. On the other hand, vacuum devices, commonly known as vacuum pumps, may be designed to create a vacuum or reduce the air pressure in an enclosed or mostly enclosed space. Thus, liquid pumps may be typically designed for liquid handling applications, whereas vacuum pumps may be typically designed to manipulate air pressure in an enclosed or mostly enclosed space. The disclosed pump module 302 may be designed to perform both functions such that the pump module 302 may reduce air pressure in the transport channel 112 as well as pump liquid from the urinary catheter 100.

In certain embodiments in accordance with the invention, the pump module 302 may be activated/deactivated (i.e., turned on and off) by a remote control 304. For example, the remote control 304 may use a radio frequency to remotely activate/deactivate the pump module 302. This may enable a user to turn the pump module 302 on or off without physically touching the pump module 302 and enable the pump module 302 to be placed at a location that is convenient and/or away from the user. When the user needs to urinate and evacuate the urinary catheter 100, the user may activate the pump module 302 using the remote control 304. When finished, the user may similarly deactivate the pump module 302 using the remote control 304. In certain embodiments, the remote control 304 includes a clip, chain, strap, band, etc. that enables the remote control 304 to be tethered to the user, such as to the wrist or clothing of the user.

Although only a male external urinary catheter 100 is shown in the illustrated system 300, the system 300 may work equally well with female external urinary catheters, except in this application the air entry module 114 may be omitted since air may enter the urine transport channel 112 through a porous matrix of the catheter itself. The pump module 302 disclosed herein may be used to not only collect urine from both male and female types of catheters, but also dispose of the urine without utilizing a cannister. This may provide a compact and streamlined device that can be used with various types of disposal areas, such as toilets, drains, septic tanks, receptacles, or even onto the ground when used in applications such as camping.

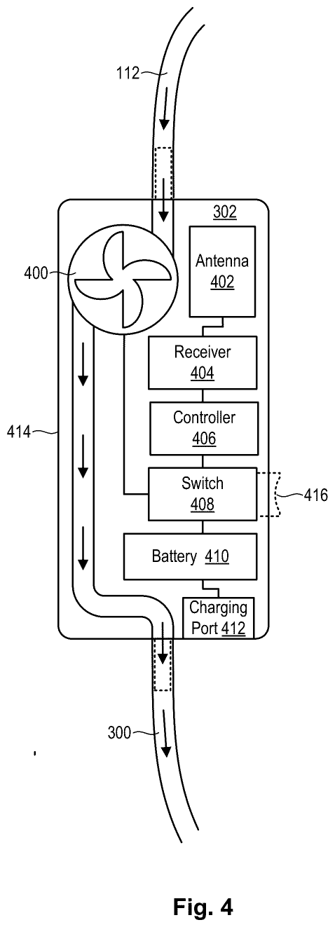

FIG. 4 is a high-level block diagram showing various exemplary internal components of a pump module 302 in accordance with the invention. Some or all of these components may be contained within a housing 414. As shown, in certain embodiments, the pump module 302 includes a pump 400 that is designed to perform dual functions, namely produce a vacuum (i.e. reduce air pressure) in the transport channel 112 as well as pump liquid from the urinary catheter 100 and create liquid pressure at its outlet. The pump module 302 may perform these functions without utilizing a vacuum cannister. Pumps that meet these requirements may in certain embodiments create a vacuum of around two meters of liquid at their inlet which is more than adequate to evacuate both male and female catheters. However, other amounts of suction are possible and within the scope of the invention.

Because a volume of air within the pump 400 may be small compared to that within a vacuum canister, suction may be created immediately within the transport channel 112 when the pump module 302 is activated. No time is needed to remove air from and generate vacuum within a cannister. This means that the pump module 302 only needs to be activated for a short amount of time during urination to evacuate the urinary catheter 100. The lack of a cannister also eliminates the need to manually and periodically empty the cannister.

As previously mentioned, in certain embodiments in accordance with the invention, the pump module 302 may be activated and deactivated using a remote control 304. Thus, in certain embodiments, the pump module 302 may include a receiver 404 to receive wireless signals from the remote control 304. In certain embodiments, an antenna 402 or other detector may be coupled to the receiver 404 to detect the incoming signals. The receiver 404 may, in turn, be coupled to a controller 406 for controlling the pump module 302 in accordance with the wireless signals. As shown in FIG. 4, the controller 406 may in certain embodiments operate a switch 408 to selectively open and close a flow of electrical current from a battery 410 to the pump 400. Alternatively, or additionally, the switch 408 may include or be coupled to a physical actuator 416 (e.g., button, rocker, toggle, slide, knob, etc.) to enable a user to manually operate the switch 408. In certain embodiments, a charging port 412 may be provided to enable charging of the battery 410 when depleted. Alternatively, the battery 410 may be disposable and replaced when depleted. Although the battery 410 is shown inside the pump module 302, the battery 410 may also be external to the pump module 302 and embodied as a separate device. The pump module 302 may also optionally be powered with cords or other non-battery power sources.

When using a controller 406 in the pump module 302, various other options for the pump module 302 are possible. For example, when the pump module 302 is activated, the controller 406 may keep the pump module 302 active for a set period of time (e.g., five seconds, ten seconds, etc.) until the urinary catheter 100 is evacuated. Alternatively, the pump module 302 may be configured to run until the controller 406 senses that the urinary catheter 100 is evacuated, such as by sensing that no additional fluids are passing through the pump 400. Various different sensors, such as flow sensors, temperature sensors, pressure sensors, fluid sensors, or the like, may be placed at strategic locations (e.g., at the urinary catheter 100, the pump module 302, etc.) and may be used by the controller 406 to determine when to deactivate the pump module 302. Similarly, in certain embodiments, sensors may also be used by the controller 406 to activate the pump module 302. For example, a temperature or flow sensor at or near the urinary catheter 100 may detect the presence of urine and automatically activate the pump 400. This may eliminate or reduce the need for a user to turn the pump module 302 on or off manually or with a remote control 304.

Similarly, in certain embodiments, the pump module 302 may not be limited to “on” or “off” but may have various different speeds. For example, a slower speed may reduce noise produced by the pump module 302. This may be helpful to operate the pump module 302 without disturbing a user or others near the user, such as when a user is utilizing the pump module 302 while sleeping or resting. Similarly, in certain embodiments, an interior or exterior of the pump module 302 may be insulated, dampened, or the like to also reduce noise produced by the pump module 302. A higher speed may increase the speed at which the urinary catheter 100 is evacuated with the tradeoff of increased noise.

Alternatively, or additionally, the pump module 302 may also be configured to run continuously without turning the pump module 302 on or off in response to urination events. This may enable the pump module 302 to run without user invention or interruption. This may, in certain cases, enable more restful sleep. For example, using this mode of operation, the suction applied to the urinary catheter 100 may be continuous and thus less jarring or interruptive than toggling the suction on and off. The pump module 302 may in certain embodiments draw very little power (e.g., some suitable pumps 400 draw around twelve watts) and thus running the pump module 302 continuously may be economical in terms of energy consumption. In certain embodiments, the pump module 302 may be configured to run in continuous mode when an outlet (i.e., grid power or mains power) is available and in intermittent mode (i.e., toggling between on and off in response to urination events using a manual switch and/or remote control 304) when the pump module 302 is running on battery power. In some embodiments, the user may choose between the two modes of operation.

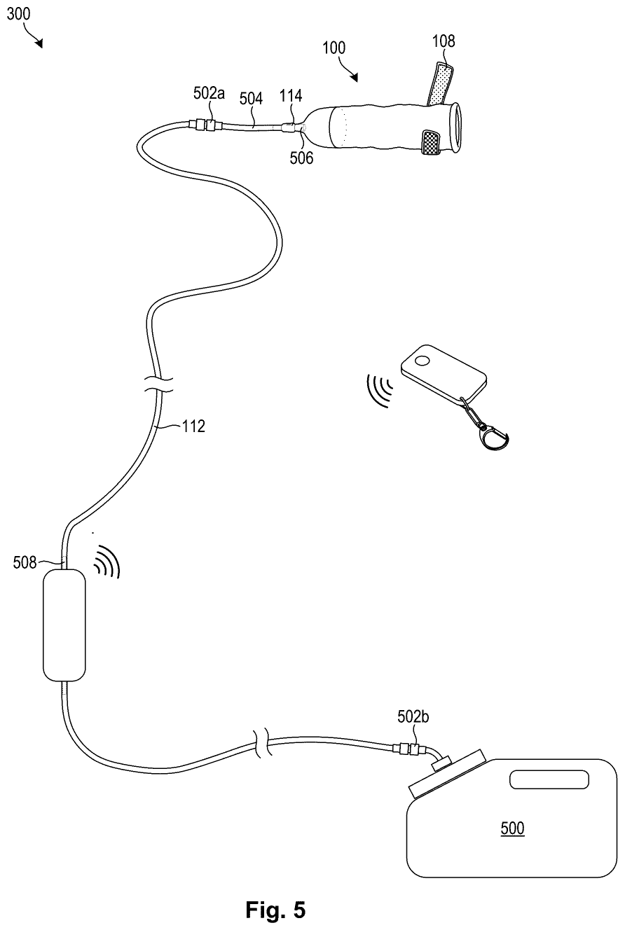

FIG. 5 is perspective view of a more specific embodiment of a system 300 including a urinary catheter 100 and pump module 302 to evacuate urine from the urinary catheter 100. In this embodiment, the system 300 evacuates the urinary catheter 100 into a receptacle 500, such as a jug with a handle, to facilitate transport and disposal. As shown, in certain embodiments, various connectors 502a, 502b, such as quick connectors 502a, 502b, may be used to connect the pump module 302 to the urinary catheter 100 and receptacle 500 respectively. When the quick connector 502a is disconnected, this may leave a short pigtail 504 of tubing on the urinary catheter 100. This piece 504 of tubing may be routed through clothing and/or clipped to a belt of the user so that it can be easily reconnected to the pump module 302 when needed and disconnected from the pump module 302 when the pump module 302 is not being used. In certain embodiments, the quick connector 502 is configured to seal off the end of the tubing 504 when in a disconnected state, thereby preventing leaking.

In certain embodiments, the receptacle 500 includes a submersible pump (e.g., a 12 volt DC submersible pump) to empty the receptacle 500 using an attached hose. However, for fixed locations such as a bedroom, the pump module 302 may create sufficient pressure to expel urine directly to a toilet or drain, thereby eliminating or reducing the need to handle urine. Practice has shown that ⅜ inch plastic tubing adhered to a bottom of a baseboard with short strips of thick clear double sided tape works well to convey urine from the pump module 302 to a toilet. Flexible silicone rubber tubing may be preferred where ninety degree bends are required. In certain embodiments, a clip may be attached to the end of a tube 304 that forms a ninety degree bend for outlet into a toilet.

In operation with one contemplated embodiment of a system 300 in accordance with the invention, the urinary catheter 100 may be attached to one end of the air entry module 114 by sliding an attachment tube of the urinary catheter 100 onto the air entry module 114 until a tip 506 of the air entry module 114 tube just enters a wider portion of the urinary catheter 100. Failure to slide it that far may leave a flexible catheter attachment tube subject to collapse and twisting under vacuum, thereby blocking the transfer of urine.

Likewise, for a similar reason, the urinary catheter 100 may be pulled onto the penis until the tip of the penis head is about halfway into the wider portion of the catheter. The penis is ideally not all the way to the end of the urinary catheter 100 as vacuum may tend to pull an end of the catheter 100 against the penis and thereby undesirably block the flow of urine. The catheter strap 108 is ideally tightened just behind the penis head to keep the urinary catheter 100 in place. This enables the use of a larger urinary catheter 100 which is typically easier to use.

The other end of the air entry module 114 may be connected to a first end of a quick connector 502a with a short piece 504 of tubing, such as ⅜ inch tubing. A second end of the quick connector 502a may be connected to tubing 112, such as ⅜ inch tubing, running to an input port 508 of the pump module 302. In use the urinary catheter 100 ideally extends through the clothing fly where, prior to urinating, it can be determined to be under vacuum by feeling that it is deflated. Also, particularly when a user is in bed, it may enable the user to verify that the tubing 504, 112 is not tangled and is running directly to the pump module 302.

During urination, the quick connector 502a is coupled and the pump module 302 is activated. Between urination events the quick connector 502a may be decoupled and the tubing pigtail 504 may be attached to a belt clip of the user, leaving the user free to walk about. When arising in the morning one procedure that works well is to turn the pump module 302 on to create vacuum while removing the urinary catheter 100 and dipping it into a container of water to rinse it. The urinary catheter 100 may then be removed and the air entry module 114 may then be immersed in the water to pull water through the system 300 and rinse it.

If desired, the urinary catheter 100 may be discarded each day and replaced with a new urinary catheter 100. However, in the absence of infection, urine is typically sterile and not the hazardous bodily fluid some have characterized it to be. By rinsing the urinary catheter 100 thoroughly each day, the urinary catheter 100 may be reused until it begins to stiffen, which is typically at least a few weeks.

In the above disclosure, reference has been made to the accompanying drawings which is shown by way of illustration specific implementations in which the disclosure may be practiced. It is understood that other implementations may be utilized and structural changes may be made without departing from the scope of the present disclosure. References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

While various embodiments of the present disclosure have been described above, it should be understood that they have been presented by way of example only and not limitation. It will be apparent to persons skilled in the relevant art that various changes in form and detail can be made therein without departing from the spirit and scope of the disclosure. Thus, the breadth and scope of the present disclosure should not be limited by any of the above-described exemplary embodiments, but should be defined only in accordance with the following claims and their equivalents. The foregoing description has been presented for the purposes of illustration and description. It is not intended to be exhaustive or to limit the disclosure to the precise form disclosed. Many modifications and variations are possible in light of the above teachings. Further, it should be noted that any or all of the aforementioned alternate implementations may be used in any combination desired to form additional hybrid implementations of the disclosure.

1. An apparatus for collecting urine from a user, the apparatus comprising:
a urinary catheter comprising an outlet to enable liquid to exit therefrom;
a transport channel having a first end coupled to the outlet; and
a cannister-free pump module coupled to a second end of the transport channel and configured to draw the liquid into the cannister-free pump module and expel the liquid into an output channel.

2. The apparatus of claim 1, wherein the urinary catheter is one of a male external urinary catheter and a female external urinary catheter

3. The apparatus of claim 1, wherein the cannister-free pump module is further configured to generate a vacuum in the transport channel.

4. The apparatus of claim 1, further comprising an air entry module, at or near the outlet, to enable air to enter the transport channel.

5. The apparatus of claim 1, wherein the cannister-free pump module is configured to be wirelessly activated by a remote control.

6. The apparatus of claim 1, wherein the cannister-free pump module is configured to be manually activated with a switch.

7. The apparatus of claim 1, wherein the cannister-free pump module is powered by a battery.

8. The apparatus of claim 7, wherein the battery is rechargeable.

9. The apparatus of claim 7, wherein the battery is incorporated into the cannister-free pump module.

10. The apparatus of claim 1, wherein the output channel empties into one of a receptacle and a toilet.

11. A method for collecting urine from a user, the method comprising:
gathering liquid with a urinary catheter comprising an outlet to enable liquid to exit therefrom;
conveying the liquid through a transport channel having a first end coupled to the outlet;
drawing the liquid through the transport channel using a cannister-free pump module coupled to a second end of the transport channel; and
expelling, using the cannister-free pump module, the liquid into an output channel.

12. The method of claim 11, wherein the urinary catheter is one of a male external urinary catheter and a female external urinary catheter.

13. The method of claim 11, further comprising using the cannister-free pump module to generate a vacuum in the transport channel.

14. The method of claim 11, further comprising enabling air to enter the transport channel using an air entry module at or near the outlet.

15. The method of claim 11, further comprising wirelessly activating the cannister-free pump module using a remote control.

16. The method of claim 11, further comprising manually activating the cannister-free pump module using an actuator.

17. The method of claim 11, further comprising powering the cannister-free pump module with a battery.

18. The method of claim 17, wherein the battery is rechargeable.

19. The method of claim 17, wherein the battery is incorporated into the cannister-free pump module.

20. The method of claim 11, further comprising emptying the output channel into one of a receptacle and a toilet.
